(19) United States
(12) Patent Application Publication
(10) Pub. No.: US 2022/0273814 A1
(43) Pub. Date: Sep. 1, 2022

(54) C60 GLUTATHIONE DOPA AND METHODS

(71) Applicant: Pharmzandia Corporation, Henderson, NV (US)

(72) Inventor: Peter Butzloff, Walnut, CA (US)

(21) Appl. No.: 17/579,967

(22) Filed: Jan. 20, 2022

Related U.S. Application Data
(63) Continuation of application No. PCT/US21/62908, filed on Dec. 10, 2021.
(60) Provisional application No. 63/154,899, filed on Mar. 1, 2021.

(51) Int. Cl. A61K 47/69; A61K 31/137; A61K 31/198; A61K 31/095

(52) U.S. Cl. CPC A61K 47/6949; A61K 31/137; A61K 31/198; A61K 31/095; B82Y 5/00

(57) ABSTRACT

A novel dual neurotransmitter nanoparticle composition is provided to store and transport protons and cations into neural cell membranes and to disassemble salt-bridge stabilized toxic protein plaques. These properties function to mitigate cognitive deficits in neurological diseases such as Parkinson's disease and Alzheimer's disease, as well as to reduce the severity of aging related reactive oxygen species damage by the sequestration and termination of free radicals and reactive oxygen species. The composition comprises C60 bonded to one or more glutathione molecules and one or more molecules of either levodopa or dopamine. The composition can be produced at low temperatures through reactive shear milling. This composition therapeutically improves and prophylactically preserves cognitive performance, memory, and mental acuity on aging to promote mental performance and health-span improvement.

### CROSS-REFERENCE TO RELATED APPLICATIONS

This application is a continuation of international application PCT/US21/62908 filed Dec. 10, 2021 which claims the benefit of and priority to U.S. provisional patent application 63/154,899 filed on Mar. 1, 2021 and entitled “MOTOR NEURON BOOSTING COMPOSITION AND METHODS” both of which are incorporated by reference herein in their entireties.

### BACKGROUND

### 1. Field of Invention

The present invention is a composition of buckminsterfullerene with reduced glutathione (GSH) and levodopa (L-dopa) pendant groups, and methods of use to prevent, or to treat, degenerative neural disease and loss of motor neuron control that is associated with or leading to neural cell death in susceptible cells. Delivery methods include ingestion, inhalation, or injection when used as a medicament or as a food supplement to maintain or re-establish benign healthy neural cellular homeostasis.

### 2. Background Art

Parkinson's disease (PD) is a movement disorder driven by the loss of dopamine producing neurons in the substantia nigra (SN) region of the human brain. PD is characterized by difficulty in initiating movement, muscle rigidity, muscle tremors, and an inability to maintain a stable posture. The motor dysfunctions represent the major clinical features of this disease. Non-motor symptoms such as sleep disturbances, dementia and depression may also be present. Motor disturbances are primarily produced by the degeneration of dopamine neurons in the substantia nigra region of the brain, as well as the projections from this region to the striatum. Additional regions of neurons may also be affected in the disease. One short term treatment strategy was based on prescribing dopamine neurotransmitter agonists. However, it was found that even when administered in combination with dietary antioxidants, dopamine promotors and dopamine itself can be ineffective or even produce negative effects after long-term administration.

Decreased levels of total glutathione (GSH) and oxidized glutathione (GSSG) with 40% to 50% deficit were found in the substantia nigra region of the autopsied brains from deceased patients with PD by Perry and colleagues in the 1980's. Later, a similar loss of antioxidant GSH in the substantia nigra was found in Incidental Lewy body disease. Lewy body disease is thought to be an early form of PD. In these cases, oxidative damage to lipids, protein, and DNA in the nigra of PD patients occurs. Past clinical trials attempting to treat PD have used antioxidants targeted to substitute for GSH, but these substances have shown little benefit, likely because of low bioavailability to the brain.

Identification of the oxidative properties of dopamine implicated it as one potential source of oxidative stress in PD when it is present under oxidative conditions. The discovery of PD-causing mutations causing alpha-synuclein aggregation in the Incidental Lewy body pathology of PD leading to neurodegeneration was later clarified by manipulation of both dopamine levels and α-synuclein expression in aged mice. It was then found that only the combination of these 2 factors caused progressive neurodegeneration of the SN region of the brain and an associated motor deficit.

Alpha synuclein in fibril form is a 14 kilo-Dalton protein. The neurodegenerative disorders associated with alpha-synuclein plaques are collectively known as synucleinopathies. The major components of intraneuronal inclusions containing alpha-synuclein plaques are termed Lewy bodies. It is now well understood that the key symptoms of PD and Lewy body disease is the aggregation of alpha-synuclein protein fibrils into pathogenic alpha-synuclein plaques.

Genetic point mutations were found to cause autosomal forms of PD by pathogenic disruption of the amino acids in the hydrophobic N-terminal domain of alpha-synuclein to cause nucleation and aggregation into β-sheet-rich plaques. Under some conditions, the neurotransmitter dopamine was able to modify alpha-synuclein aggregation in the SN, resulting in greater abundance of alpha-synuclein oligomers and dopamine-induced plaques. Furthermore, it was found that by disrupting the dopamine-α-synuclein interaction under these conditions, dopaminergic neural degeneration was reversed. Equally important and extensive evidence from cultured cells and cell-free solution testing indicates that the neurotransmitter dopamine may inhibit the aggregation of alpha-synuclein. At present, L-dopa, a dietary supplement that metabolizes into dopamine neurotransmitter, is the most widely used and most effective therapy for PD. Elevating dopamine levels under either oxidizing or reducing conditions either increased or reduced the number of cells containing insoluble and large intracellular alpha-synuclein plaques. This demonstrated that the interaction of dopamine and alpha-synuclein is noncovalent and reversible, and it also demonstrated that reduction-oxidation (REDOX) conditions reversed the conditions associated with PD. Oxidized dopamine promotes the formation of alpha-synuclein plaques that become neurotoxic in large concentrations. This pathology is caused by disrupting cytoskeletal organization in neural lipid membranes that reduce synaptic terminal membrane permeability and affects neural signaling by blocking calcium ion transport.

In more detail, it is now clear that healthy individual alpha-synuclein fibrils in neurons bond with the inner (cytosolic) leaflets of the presynaptic membrane leaflet to induce a concentration dependent stabilization of docked and double-anchored synaptic vesicles. Neurodegenerative oxidative and free radical damage forms cross-links within and thickens the overall lipid composition of the cellular membranes. This chemical change allows alpha-synuclein to bond to outer presynaptic membranes and disrupts the correct morphology and conformation of alpha-synuclein, encouraging the formation of plaques. The movement of toxic plaques to the inner presynaptic membranes then causes dysfunctional disruption of synaptic vesicle membrane transport.

On a related topic, glutathione (GSH) is the most abundant small molecule, non-protein thiol in cells. It consists of a tripeptide of glutamate, cysteine, and glycine. Within neurons, GSH is found in greatest abundance at nerve endings. It is now well understood that GSH in the neural synapse serves as a REDOX regulator that functions to control and maintain intracellular REDOX. Moreover, the absence of GSH in the substantia nigra region of the brain leads to dysregulated conditions that promote neurotoxic alpha-synuclein plaque formation. The degeneration of dopaminergic neurons in the substantia nigra during PD is therefore directly related to GSH depletion that leads to elevated levels of nitric oxide and peroxynitrite oxidants, leading to oxidative stress damage. This damage inhibits the REDOX complexes of the electron transport chain, causes a drop in the proton motive force, and reduces ATP production to further magnify the REDOX dysfunction by causing mitochondria to significantly reduce GSH synthesis. The further loss of GSH increases oxidative and free radical damage to surrounding cellular lipids and increasingly creates those toxic conditions leading to neurodegeneration.

Given these understandings of the etiology and neuronal disease characteristics associated with the progression of PD, designs of materials or compositions able to decelerate or alleviate PD and related motor neuron disease progression is now better focused, and the targets of oxidative stress and plaque formation have achieved great importance. In particular, the anti-aggregation effect of fullerenols, previously demonstrated for various forms of amyloid proteins, was also observed to be somewhat effective for the reduction of alpha-synuclein insoluble protein aggregates. The limited fullerenol neuroprotective activity was not affected by the number of carbon atoms in the fullerenol compounds, so that polyhydroxylated C60 or C70 was able to act equally well. While fullerenols might be a promising tool for drug development to treat PD, these materials are also understood to be insufficiently targeted to the specific regions of neuronal dysfunction, such as the SN region of the PD brain, and do not have an established ability to cure or prevent PD.

The consensus among scientists and medical practitioners is that oxidative stress plays a major role in the development of neural cellular metabolic disease. Some of the current therapeutic strategies have put emphasis on the design of multiple functional properties into molecules or particles that enable them to target multiple enzymes or receptors, allowing them all to operate at the same time to avoid or correct the dysfunctions leading to disease states. Of these, several natural antioxidants have been known for their ability to trap and scavenge free radical species and reactive oxygen species (ROS) such as hydroxyl and nitrate free radicals. ROS are associated with causing damage to essential cellular components, and neurodegenerative diseases.

While there is great promise in the widely known beneficial neuroprotective functions of glutathione in the brain, there are no known strategies to enhance its delivery into those tissues that do not produce this substance. Therefore, new methods are being sought to deliver this substance to the substantia nigra portion of the human brain, where the endogenous production of certain substances like glutathione has failed locally in the Parkinson's patient. One important function of glutathione is to bind and activate the ionotropic receptors, potentially making it a neurotransmitter. However, the evidence for glutathione being a neurotransmitter is sparse and controversial because it also induces calcium signals and the release of gamma amino butyric acid (GAB A) from some types of neurons and glia. It has been difficult to disentangle the neurotransmitter role of GABA from that of glutathione. It happens that this is a useful observation because it may be possible to operate on glutathione to effect remedial change in the release of GABA. GABA is the principal inhibitory neurotransmitter in the mammalian central nervous system. Dysregulation of GABA is already implicated in many neurological disorders, such as in Alzheimer's disease, epilepsy, panic disorder, and anxiety, whereas dysregulation of glutathione is implicated in the etiology of Parkinson's disease and autism. Thus, it is of essential medical utility to carefully examine the multiple dimensions of interaction of glutathione as a master regulatory substance.

A limitation to the use of supplemental glutathione to improve neurological health, is in the lack of maturity of cell signaling designs. Such design failures are attributed to an incomplete understanding of cell signaling functions and protein messaging effects that are part of motor neuron disease development. Cell signal interactions begin with surface charges at membranes. Surface charges are in contact with the cell cytosol, proteins, deoxyribonucleic acids (DNA), and the lipid membranes of the cell. Some signaling regions, such as the endoplasmic reticula of mitochondria, may become insufficiently engaged in reduction-oxidation that is associated with the development of neurological cell death. This deficit, along with reactive oxygen species (ROS) associated in the aging process, are thought to contribute to dysfunction of the electron transfer cycle that allows proper cellular respiration to take place, and the result can be the production of misfolded proteins associated with neuronal toxicity and cell death. Unfortunately, the present trend in the development of improved glutathione supplements or glutathione-based pharmaceuticals and nutraceuticals have demonstrated limited or no control over cell signaling, and there has not been a successful composition or method to improve neurological health in Parkinson's disease patients using glutathione or variations of glutathione.

Dopamine agonists are state of the art medications based on a derivative of dopamine that have been medically proven to stimulate the parts of the human brain influenced by dopamine. The neurons of the brain, especially in the substantia nigra where motor control is interfaced with control signals propagating into and from the brain stem, can accept these exogenous, artificial, and introduced dopamine substitutes. The neurons are then able to perform and function as if accepting the endogenous dopamine that these neurons need but have not been able to receive in sufficient quantity over the early stages of the neurological disease. In general, dopamine agonists are not as potent as carbidopa or levodopa and may be less likely to cause dyskinesias. However, a better solution is not commercialized yet, and most people who take levodopa develop motor control problems within 5 to 10 years after starting their dopamine agonist medicine. These complications are unpredictable swings in muscular position control between doses and uncontrollable jerking or twitching (dyskinesias). Dyskinesia can become so intense that that it is as disabling as some of the problems caused by the neurological disease. In the longest study done, people who started treatment with a dopamine agonist had just as many problems with motor fluctuations at 14 years as people who started treatment with levodopa, indicating a failure to prevent oxidative damage at the post synaptic bouton, a failure to protect neurons from cell death, and a failure to prevent the accumulation of toxic brain protein oligomers such as alpha-synuclein plaques that are a cause of neural cell death. The study of Olanow (2013) published that those observed factors most predictive of developing dyskinesia from L-dopa and agonists of dopamine in combination with L-dopa. These predictive results are ranked order from most to least, being: (1) young age at onset, (2) higher L-dopa dose, (3) low body weight, (3) North American geographic region, (4) The L-dopa with carbidopa and entacapone (two agonist drugs) treatment group, (5) female gender, and (6) a more severe Unified Parkinson's Disease Rating Scale (UPDRS) Part II. There is clear medical benefit, medically verified neuroprotective effects, and no known toxicity of L-dopa administered either at early stages or at late stages of Parkinsonism. However, there can also be side effects from L-dopa treatment either in combination with or without dopamine agonists: these side effects may include gastrointestinal disturbances, cognitive problems, and sedation. The more recently approved combination therapies of L-dopa with the newer dopamine agonist drugs (also known as dopaminergic substances) still cause dyskinesia in late-stage Parkinson's disease, where this problem persists to the modern day, in the state of the art; a study by Shiraishi (2019) is one such example.

The severe economic impact to families and society from insufficiently comprehensive state-of-the-art neural disease treatments for conditions such as Parkinson's disease remains a burden that society can ill afford. Moreover, the advent of COVID-19 and mutated novel coronavirus has led to a rise in the incidence of Parkinsonism in many affected individuals.

What is therefore needed is a novel therapeutic strategy or unique material used to confer improved cellular neuron protection and significantly prevent, mitigate, or reverse toxic pathology arising from synaptic and neurological dysfunction before irreversible damage progresses. Desirably, such a treatment should prevent or avoid dyskinesia by including a means to remove sources of oxidation and free radical generation, to include a very localized and very targeted remediation of reactive oxygen species. It is believed the present invention provides a broadly effective discovery of such a composition, having an intelligent biological and electrochemical design incorporating glutathione to confer multiple therapeutic and prophylactic functions that are highly targeted to neural synaptic structures, especially those in the substantia nigra of the brain. This novel composition will change our perspective on applications to boost resistance and generate recovery to the effects of Parkinson's and other neurological disease. The use of traditional carrier formulations will enable appropriate methods of administration for this novel composition.

### SUMMARY OF THE INVENTION

This invention is a cluster of nanoparticles composed with fullerene dopamine glutathione material with tremendous advantages that behaves in unique manners to treat and proactively reduce the conditions leading to neurological disease such as Parkinson's Disease, amyotrophic lateral sclerosis, Alzheimer's disease, and other pathological neuron damage. This composition possesses a combination of properties targeted to those plaque forming regions which require salt-bridge disruption and free radical scavenging enabled by fullerenes such as buckminsterfullerene (C60), the antioxidant properties of a glutathione functional group deliberately carried and targeted to regions where dopamine is utilized at the desired neural structures, a storage reservoir to provide reducing hydrogen protons to confer localized chemical reducing conditions, and the provision of a plaque disassembly function of metabolized L-dopamine functional groups that have become decarboxylated to form dopamine functional groups at such locations.

In one aspect, the C60-GSH-DOPA composition protects and enhances the membrane polarization of mitochondria by being able to penetrate them and protect them from oxidative stress. This allows protected mitochondria to significantly enable their normal function and undisrupted ability to generate reducing protons, where such hydrogen protons are then able to achieve reducing REDOX conditions. This nanoparticle molecular structure possesses charge storage properties targeted to break plaque forming regions using a salt-bridge disruption technology. The free radical scavenging and targeted delivery to brain neurons is also enabled. The antioxidant properties of the functional groups are deliberately carried to oxidatively stressed regions where dopamine and glutathione (GSH) are utilized at the desired neural structures, being the post synaptic bouton, while also providing a storage reservoir of reducing hydrogen protons on the C60 and the amine functional groups to confer a localized chemical reducing condition. The provision of the plaque disassembly function provides accelerated cation trafficking functionality needed by neurons at the synapse.

In a key functional aspect, C60-GSH-L-Dopa and its metabolites to C60-GSH-DOPA provide an artificial pathway to supplement and accelerate the trafficking of cations for proton exchange to prevent or remove salt accumulation among oligomeric fibrils. This function acts to disassemble the oligomeric plaques formed by salt cations by extracting these cations, so that they may not serve as salt bridges. This aspect of the invention depends on the use of the zwitterionic properties of the nanoparticle functional groups. C60 is normally considered anionic when it collects as many as six negative charges. The association of C60 with zwitterionic functional groups has the additional properties of being an organic salt, in which both hydrogen bonding as well as aromatic pi to cationic pi bonding contributes to the stability of these structures and defines how this collective ensemble serves to traffic both protons and physiological cations such as potassium and sodium.

In another aspect, the C60-GSH-DOPA composition protects and enhances the membrane polarization of mitochondria by being able to penetrate them and protect them from oxidative stress. This allows protected mitochondria to significantly enable their normal ATPase function and undisrupted ability to generate reducing protons, where such hydrogen protons are then able to achieve reducing REDOX conditions at the neural post-synaptic terminal.

In a related aspect, the composition of this invention accrues and transports hydrogen protons to regions removed from the mitochondria where protons are required to exchange for physiological cations such as potassium, and sodium. This aspect can supplement endogenous substances fulfilling the same role.

In a related aspect, the free radical protective effect of the C60-GSH-DOPA on mitochondria ensures the uninterrupted mitochondrial provision of chemically reductive protons. In a cascade effect, the produced protons act directly on dopamine molecules to enable them to maintain healthy individual alpha-synuclein fibrils in neurons. The functional individual alpha-synuclein fibrils then bond with the inner (cytosolic) leaflets of the presynaptic and post-synaptic membrane leaflets to stabilize the functional release and reacquisition of synaptic vesicles on neurostimulation.

In a key aspect, the technological hurdle of supplying exogenously produced GSH neurotransmitter to the brain is provided by using a buckminsterfullerene (C60) carrier to enable crossing of the blood brain barrier and allow glutathione's well known and outstanding medical benefits, including reducing blood pressure and enhancing long-term memory, to be directly promoted to each brain region and all brain tissues.

In a related aspect, the transport of GSH into the brain by C60 allows it to be protected by the C60 functional group so that the adduct of GSH is unable to be easily broken down by neural enzymes, especially those released by astrocytes. This enhanced stability promotes the circulation of GSH as a functional group with an extended lifetime or residence, in which it acts as both an antioxidant and as a critically important neurotransmitter.

In another aspect, the provision of the GSH functional group on the C60-GSH-DOPA is to substitute for a lack of endogenously produced glutathione (GSH) antioxidant in mitochondria. This replacement is neuroprotective to the mitochondria and acts to enable the ability of the mitochondria to return to a state of homeostasis, where it can now recycle the nanoparticles as modified exogenous neurotransmitters for release. The exogenously produced C60-GSH-DOPA may then bond with the lipids of the cellular membranes, including lipids at the outer (intracellular) presynaptic membrane leaflets to act in like manner to missing glutathione, as a reducing agent to prevent the accumulation of free radicals and oxidative damage to membrane lipids. This protection thereby prevents alpha-synuclein from otherwise forming toxic plaques by cross-linking reactions.

In another aspect, the presence of C60-GSH-DOPA is to penetrate those locations in the neural structures already biochemically attractive to dopamine. The dopamine functionality of the introduced C60-GSH-DOPA is otherwise identical to and complementary with that of native or endogenous dopamine neurotransmitter. The advantages of this targeted delivery system are the highly localized delivery of GSH functionality as well as that of the fullerene groups to provide free radical quenching and powerful antioxidant functions to the lipid surfaces to those oxidative locations where dopamine is required for proper neurotransmission, but in which GSH normally does not migrate, and in which C60 is never found except when externally provided.

The result of these combined functions is to allow time for the proper re-integration of dysfunctional or senescent neural cells by introducing an enhanced REDOX reversibility, and to directly inactivate reactive oxygen species (ROS) in mitochondria and at the surface membranes of cellular organelles to re-establish functional cellular homeostasis.

In a related aspect, the function of the antioxidant fullerene glutathione dopamine (C60-GSH-DOPA) is to correctively interact with alpha-synuclein oligomers arising from the otherwise pathological interaction with ordinary dopamine under dysregulated and oxidizing conditions. Thus, C60-GSH-DOPA functions as a dopamine mimetic, being functionally identical to dopamine, and taking part in the same biochemical reactions as dopamine yet providing localized therapeutic reducing conditions critical to regulating neural cell function and restoring healthy neurotransmitter signaling at the synapse.

In yet another aspect, C60-GSH-DOPA disrupts sodium ion salt bridges between plaque fibrils to return individual strands of alpha-synuclein fibrils to their proper conformation and neurological function. Largely, it is the presence of the core fullerene molecule being tethered to a dopamine functional group that helps to disassemble detrimental salt bridges between proteins. The high negative charge density acquisition of the fullerene group enables the abstraction and sequestering of sodium cations onto itself and away from the plaque proteins.

In another aspect, the C60-GSH-DOPA provides free radical quenching and antioxidant effects together with free radical recombination via the combined activity of both the GSH functional group and the fullerene C60 group, thereby ensuring a reducing rather than oxidizing role in the presence of the metabolized dopamine functional group in this composition, to deter the formation of alpha-synuclein plaques, and to substantially avoid oxidized dopamine release of hydrogen peroxide to inflict damage on neural tissues.

In another aspect, the C60-GSH-DOPA composition is formulated to allow it to become sequestered into the pores of food grade Transcarpathian zeolite (clinoptilolite) for the purpose of timed-release delivery of the orally administered composition.

In another aspect, the C60-GSH-DOPA composition is administered in the form of a nano-aerosol for the purpose of immediate aspirated delivery to the lungs, thereby providing more direct access to the blood system for rapid release of the administered inhalant composition to the brain and bypassing the digestive system as well as any oxidative damage incurred by the digestive tract fluids to the composition.

These and other advantages of the present invention will be further understood and appreciated by those skilled in the art by reference to the following written specification, claims, and appended drawings.

Some embodiments are described in detail with reference to the related drawings. Additional embodiments, features, and/or advantages will become apparent from the ensuing description or may be learned by practicing the invention. In the illustrations, which are not drawn to scale, like numerals refer to like features throughout the description. The following description is not to be taken in a limiting sense but is made merely for describing the general principles of the invention.

### BRIEF DESCRIPTION OF THE DRAWINGS

Preferred embodiments of the invention will now be described, by way of example, with reference to the accompanying drawings, in which:

FIG. 1 is an illustration of some molecular structures of raw materials relevant to the teachings of the present invention.

FIG. 2 is an illustration of molecular structures of the reversible REDOX reaction of glutathione (GSH).

FIG. 3 is an illustration of molecular structures of the reactions of glutathione (GSH) with buckminsterfullerene (C60).

FIG. 4 is an illustration of the molecular structures of the reactions of levodopa (L-dopa) with buckminsterfullerene (C60).

FIG. 5 is an illustration of dopamine, glutathione, and C60 chemically reacting to synthesize C60-GSH-Dopa having multiple aryl pi-pi bond formation.

FIG. 6 is an illustration of C60-GSH-L-dopa conformations in which pi-carbonyl bonds, aromatic pi to aromatic-pi bonds, and zwitterionic hydrogen bonds create a molecular network structure.

FIG. 7 is an illustration of alpha-synuclein plaques being intercalated with and disassembled by clusters of C60-GSH-L-dopa and/or metabolites thereof comprising C60-GSH-DOPA.

FIG. 8 is an illustration of clusters of C60-GSH-L-dopa and/or metabolites thereof comprising C60-GSH-DOPA providing protection and treatment at the neural synapse and at neural membranes.

FIG. 9 is an illustration of a molecular structure for Transcarpathian zeolite (clinoptilolite) binder permeated or filled with C60-GSH-L-dopa.

FIG. 10 is a flowchart representation of a synthesis of C60-GSH-L-dopa with a formulation for use as a nano-aerosol inhalant.

FIG. 11 is a flowchart representation of a synthesis of C60-GSH-L-dopa with formulations for Oral Administration.

FIG. 12 is an illustration of personal administration of aspirated nano-aerosol C60-GSH-L-dopa.

FIG. 13 is an illustration of experimental FTIR data for levodopa (L-dopa).

FIG. 14 is an illustration of experimental FTIR data for buckminsterfullerene levodopa (C60-L-dopa).

FIG. 15 is an illustration of experimental FTIR data for reduced glutathione (GSH).

FIG. 16 is an illustration of experimental FTIR data for buckminsterfullerene glutathione (C60-GSH).

FIG. 17 is an illustration of experimental FTIR data for C60-GSH-L-dopa.

FIG. 18 is an illustration of an experimental negative mode mass spectrograph data for C60-L-dopa.

FIG. 19 is an illustration of an experimental negative mode mass spectrograph data for C60-GSH.

FIG. 20 is an illustration of an experimental negative mode mass spectrograph data for C60-GSH-L-dopa.

Some embodiments are described in detail with reference to the related drawings. Additional embodiments, features, and/or advantages will become apparent from the ensuing description or may be learned by practicing the invention. In the illustrations, which are not drawn to scale, like numerals refer to like features throughout the description. The following description is not to be taken in a limiting sense but is made merely for describing the general principles of the invention.

### DETAILED DESCRIPTION OF THE INVENTION

The following detailed description, taken in conjunction with the accompanying drawings, is merely exemplary in nature and is not intended to limit the described embodiments or the application and uses of the described embodiments. Any implementation described herein as “exemplary” or “illustrative” is not necessarily to be construed as preferred or advantageous over other implementations.

Furthermore, there is no intention to be bound by any expressed or implied theory presented in the preceding technical field, background, brief summary or the following detailed description. It is also understood that the specific devices, systems, methods, and processes illustrated in the attached drawings, and described in the following specification, are simply exemplary embodiments of the inventive concepts defined in the appended claims that there may be variations to the drawings, steps, methods, or processes, depicted therein without departing from the spirit of the invention. All these variations are within the scope of the present invention. Hence, specific structural and functional details disclosed in relation to the exemplary embodiments described herein are not to be interpreted as limiting, but merely as a representative basis for teaching one skilled in the art to variously employ the present embodiments in virtually any appropriate form, and it will be apparent to those skilled in the art that the present invention may be practiced without these specific details.

Various terms used in the following detailed description are provided and included for giving a perspective understanding of the function, operation, and use of the present invention, and such terms are not intended to limit the embodiments, scope, claims, or use of the present invention.

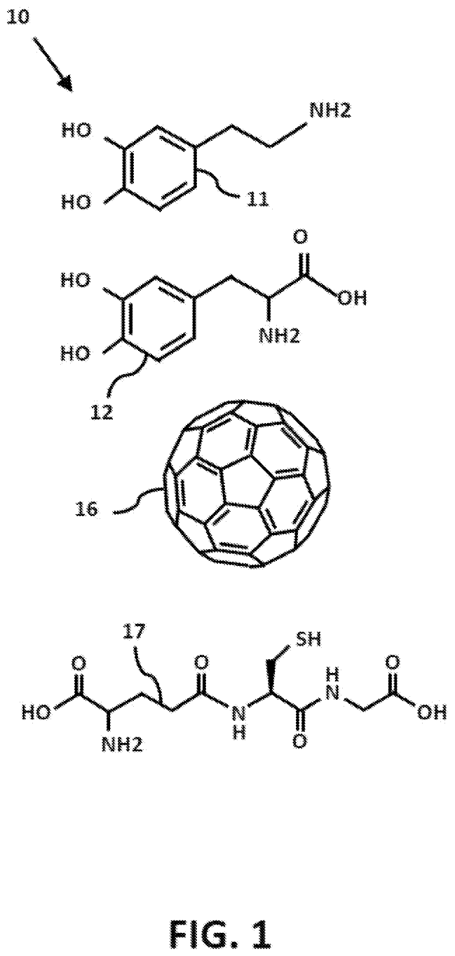

FIG. 1 illustrates molecular structures 10 used or metabolized in the composition of the present invention. Dopamine (DOPA) 11 has chemical formula C₈H₁₁NO₂ and is also known as the endogenous neurotransmitter 3,4-dihydroxyphenethylamine. Levodopa (L-dopa) 12 is an amino acid of chemical formula C₉H₁₁NO₄ that is commercially available as a synthetic food supplement and is readily metabolized by decarboxylation to form the neurotransmitter dopamine (DOPA) 11 as well as other neurotransmitters. It is generally understood and recognized that L-dopa 12 is a chief chemical precursor to DOPA 11 and may be used in neuroprotective treatments for Parkinson's Disease and other neurological disorders. The molecular structure 17 is reduced glutathione and has the chemical formula C₁₀H₁₇N₃O₆S. GSH may function somewhat as a neurotransmitter in that it operates on GABAergic neurons to release gamma amino butyric acid (GABA) and may have other endogenous signaling functions. Buckminsterfullerene 16 is a single molecule comprised of 60 carbon atoms arranged as a sphere and has the chemical formula of C60. Substances 11, 12, 13, 17 may be used to help create, process, or deliver parts of the composition of C60-GSH-L-dopa.

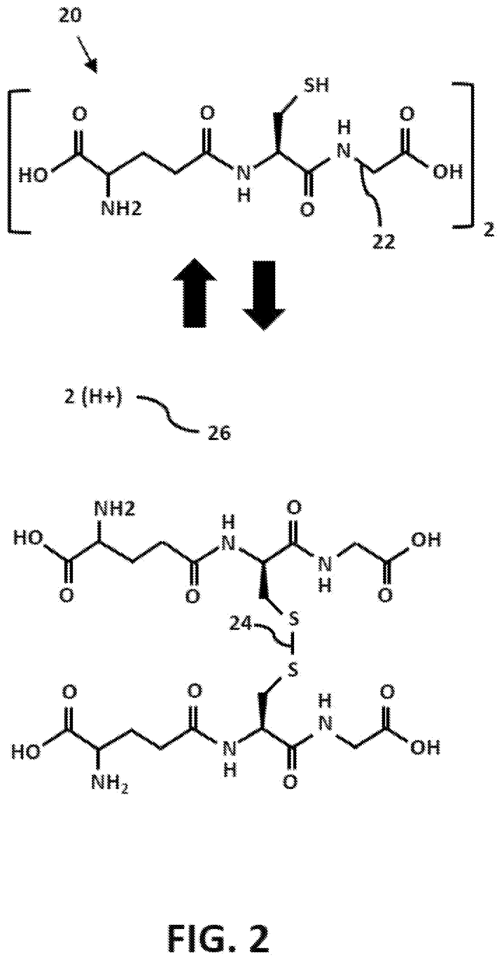

FIG. 2 illustrates the molecular structures of the reversible biochemical oxidation reaction of glutathione (GSH) 20. Two of the reduced form of glutathione molecules 22 become oxidized into a dimeric form of glutathione having a characteristic sulfur to sulfur bond 24. In the biochemical process of cellular respiration, the oxidized form of glutathione 24 (also known by the abbreviation GSSH), is reduced by two hydrogen protons 26 to reform GSSH into two discrete GSH molecules 22. It is understood that this is a reversible biological oxidation and reduction (redox) process as indicated by the directions of the upward and the downward solid black arrows. Reversible REDOX reactions will likewise take place with the GSH functional groups of the various derivatives of glutathione specified herein.

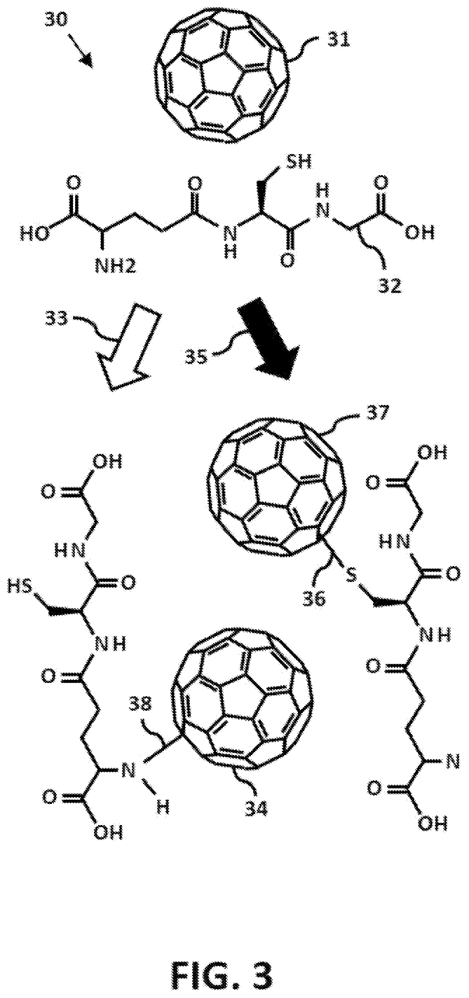

FIG. 3 illustrates molecular structures of two chemical reaction pathways 30 of glutathione (GSH) 32 with buckminsterfullerene (C60) 31. Hydrogen bonds are indicated by dotted lines and pi-bonding is indicated by dashed lines herein and throughout this specification. At elevated temperatures being above about 50° C., the direction of the reaction pathway increasingly follows the white arrow 33 to produce at least one covalent bond 38 between the at least one GSH nitrogen functional group and the C60 functional group to form a covalently bonded GSH-C60 34. This high temperature reaction pathway is undesirable because it removes the neuroprotective and antioxidant effect of the nitrogen amine functional group. At room temperature being below at most 40° C., the direction of the reaction pathway under high pressure shear conditions, substantially follows the solid black arrow 35 to produce at least one sulfide bond 36 between the at least one GSH and the C60 functional group, forming the configurational isomer of GSH-C60, 37 having the preferred geometry in which the amine nitrogen of GSH is free to act as a reducing agent against oxidants in a neuroprotective manner.

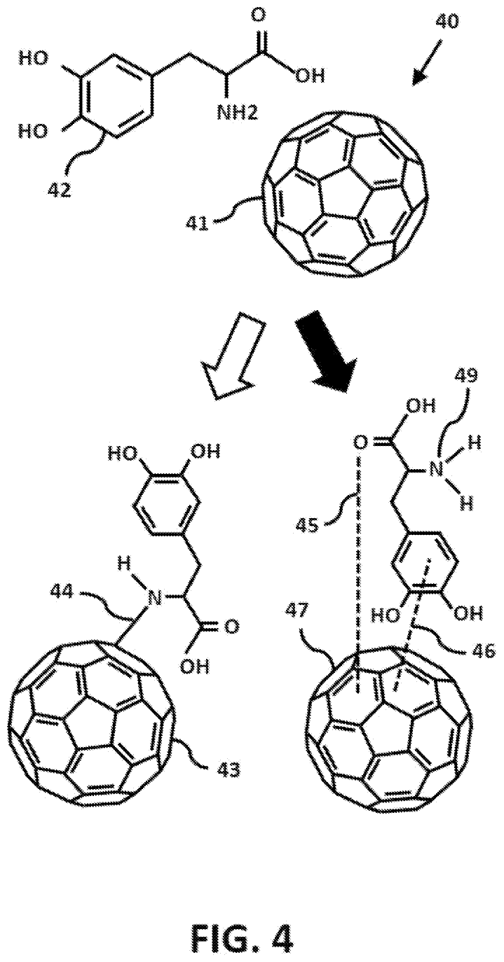

FIG. 4 illustrates molecular structures of two chemical reaction pathways 40 of L-levodopa (L-dopa), 42 with buckminsterfullerene (C60), 41. At elevated temperatures being above 120° C., the direction of the reaction pathway follows the white arrow to produce at least one covalent bond 44 between the at least one L-dopa nitrogen functional group, or at the carboxylic acid functional group, to react with a carbon atom in C60 43; this type of reaction is to be avoided at the C60 functional group, because these two covalently bonded configurational isomers of L-dopa do not ensure the preservation of a labile and neurologically available amine adduct in accordance with the molecular design specified herein. The pi bonded L-dopa with C60 is capable of being achieved under shear mixing conditions and at room temperature or below at most about 40° C. Pi bonds are stronger than hydrogen bonds, but much weaker than covalent bonds; this also means that they can form with less energy. The low temperature and high shear pressure reaction is in the direction of the reaction pathway that follows the solid black arrow to produce aromatic pi to carbonyl bond 45 and/or an aromatic-pi to aromatic-pi bond 46 between the at least one GSH functional group and the C60 functional group, being C60-Ldopa 47, having the preferred adduct geometry in which the amine nitrogen of the L-dopa functional group 49 is free to attract a hydrogen proton to act as a reducing agent against oxidants in a neuroprotective manner.

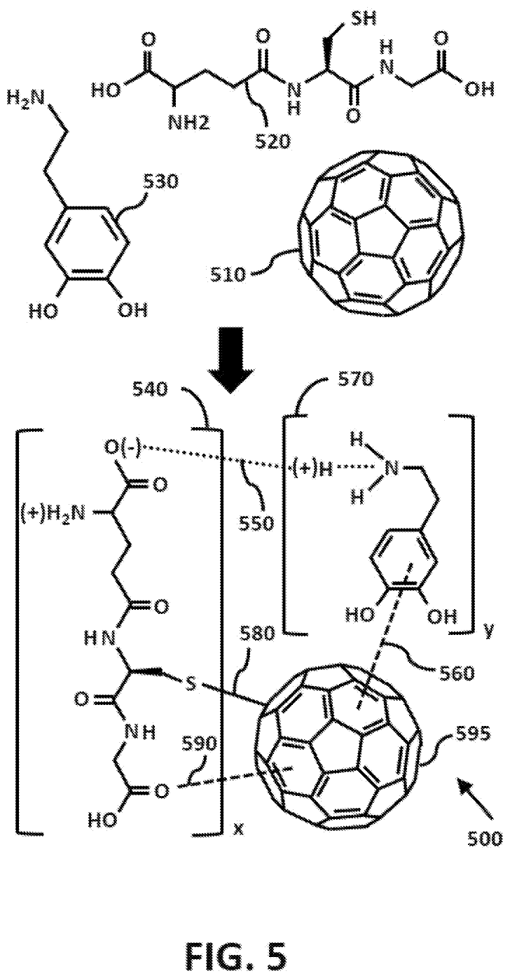

FIG. 5 illustrates the chemical reaction 500 of dopamine (Dopa) 530 and glutathione (GSH) 520 with buckminsterfullerene (C60) 510, to generate the products shown in the direction of the large black arrow. It is understood that metabolic conversion of L-dopa to dopamine 530 occurs by loss of the carboxyl (—COOH) functional group from levodopa, therefore the use of dopamine as a starting material is interchangeable with, and functionally equivalent to, the use of L-dopa in the present invention and is hereby explicitly specified. The multiplicity of functional groups of GSH denoted by the subscript letter x after the molecular structure within the bracketed region 540, is shown covalently bonded through sulfur to C60 595. The multiplicity of Dopa functional groups 570 denoted by the subscript letter y after the molecular structure within the bracketed region, may become reversibly hydrogen bonded to any GSH 540, 520 though a hydrogen bond 550. Nominally, x is 1 and y is 2, where it is understood that the neurotransmitter Dopa functional groups 570 can be replaced by levodopa as these will become metabolized to Dopa. Aromatic pi-to aromatic-pi bond represented by dashed line 560 and aromatic pi to carbonyl bond 590 each have more molecular structural strength than a hydrogen bond 550 but are less strong than a covalent bond such as bond 580. Temperatures above 55° C. tend to form the sulfur covalent bond 580, however the formation of pi-carbonyl bonds 590 may form in preponderance without covalent bonding 580 at reaction temperatures below about 40° C. Both GSH 520 and dopamine 530 function as independent neurotransmitters, however it is the design of the present composition 500 to promote these as a dual neurotransmitter function of functional groups GSH 540 and Dopa 570, thereby conferring oxidation resistance to those regions of the neuron such as the post-synaptic bouton where the absorption of functional group dopamine 570 coupled with GSH acts to promote neuronal healing and recovery from the neurological damage of Parkinson's disease and other neurological pathology.

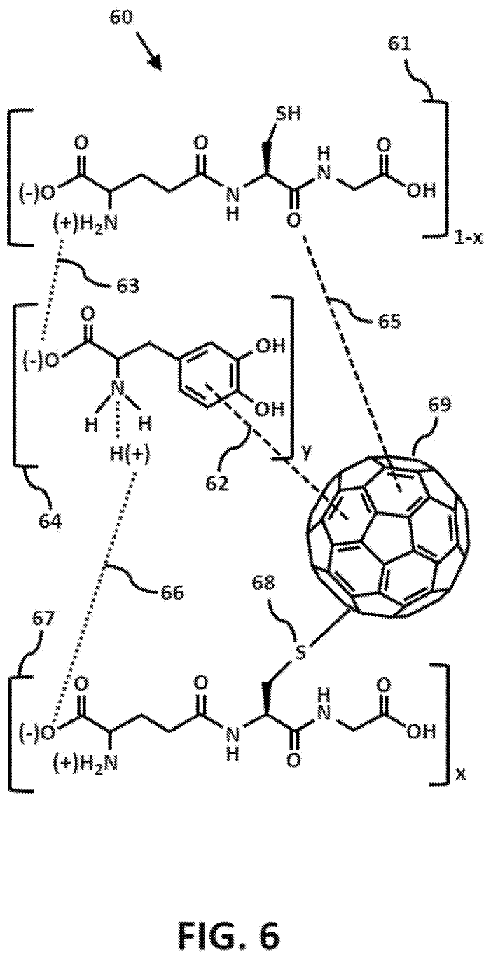

FIG. 6 illustrates the molecular structures leading to formation of a networked C60-GSH-L-Dopa 60. It is understood that dopamine will be formed after decarboxylative metabolism of some or all the L-dopa functional groups 64. Administering L-dopa alone can lead to excessive undesirable neural signaling and may also cause many of the adverse side effects associated with Dyskinesia under conditions of oxidative stress by means of the networked molecular structure 60 promoting neuroprotection by means of the antioxidant buckminsterfullerene (C60) group 69 and the antioxidant reduced glutathione (GSH) groups 61, 67. A multiplicity of hydrogen bonds is represented by dotted lines 63, 66 in these structures. A multiplicity of pi-bonds is illustrated as dashed lines 62, 65 extending outwards from the C60 group, by representative pi-carbonyl 65, and pi-pi bonds 62. Levodopa (L-dopa) 64 is an amino acid of chemical formula C₉H₁₁NO₄ that is commercially available as a synthetic food supplement. Each levodopa (L-dopa) 64 functional group on C60, 69 is readily metabolized by decarboxylation to form dopamine (DOPA). Glutathione 61, 67 is considered a neurotransmitter as well as an antioxidant. It can reversibly bond through the sulfur atom to C60 at 68, or it can bond by pi-carbonyl bond to C60 at 61, where the latter pi-carbonyl molecular configuration 61 allows the reducing power of the sulfur group (sulfhydryl) to perform as a reducing agent.

At least one glutathione (GSH) molecule 61 has formula C₄H₉NO₂ and reacts with a multiplicity (z) with C60 69. At least one and as many as about 8 (y=1 to 8) of the levodopa functional groups 64 reacts with C60, 69. Sometimes, GSH 67 can react through the sulfur group 68 to form the derivative C60-GSH providing a multiplicity of GSH functional groups. Both GSH and L-dopa form zwitterions at physiological pH of 7.3, and as C60 is normally considered anionic when it collects as many as six negative charges, the association of C60 with these zwitterions has the properties of being an organic salt, in which both hydrogen bonding as well as aromatic pi bonding contribute to the stability of these structures. Composition variations may be tuned by the number but not the type of functional groups, depending on penetrating and trafficking function, and may be from at least one L-dopa to about 6 L-Dopa, in which C60 bonded with 2 DOPA and 1 GSH functional groups promote adequate and sufficient medical improvement in human Parkinson's disease case studies. The fully decarboxylated metabolite C60-GSH-DOPA promotes therapeutic neuroprotective and neurogenesis functions, according to the teachings of the present invention.

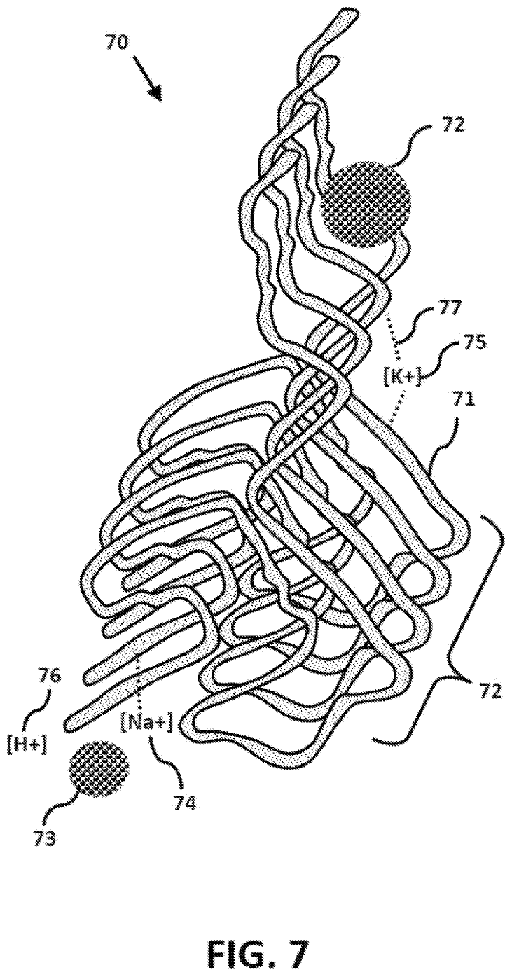

FIG. 7 illustrates the role of metabolized C60-GSH-DOPA to disassemble the toxic oligomeric plaque of alpha-synuclein 70. A substantially one-dimensional fibril of alpha-synuclein 71 tends to form lengthwise abutting bonds with a multiplicity of other alpha-synuclein fibrils termed more generally an oligomeric plaque 72. The type of bonding along adjacent fibril lengths can include van-der-Waals induced charges, however salt cations such as sodium 74 may also intercalate or squeeze between these fibrils to create tangles that increase in size with time; oxidative species may additionally interpose cross-links and protein functional groups into random locations of the alpha-synuclein fibrils to include aldehydes or carboxylic acids under oxidative conditions. Free radical additions may also form bonds between fibrils when free radicals are present.

The introduction of clusters containing C60-GSH-DOPA 72, 73 into and among alpha-synuclein plaques 72 allows the quenching of free radicals and provides anti-oxidant functionality. Clusters containing C60-GSH-DOPA 72, 73 also store and then release hydrogen protons 76 carried at the amine nitrogen of dopamine functional groups, in which up to about five additional hydrogen protons may be carried by the fullerene C60 functional group. Fullerenes are also known for their ability to store as many as six (6) negative charges, whereby the high negative charge concentration in the clusters of C60-GSH-DOPA 72, 73 can extract sodium cations 74 from plaque 72, thereby freely releasing individual alpha-synuclein fibrils 71 from the collective plaque tangle 72. The combination of free-radical quenching, anti-oxidant function, cationic extraction, and free proton release enables the proper function of dopamine neurotransmitter. The targeting of reductive DOPA functional groups from C60-GSH-DOPA to those oxidative locations at the post-synaptic terminal counteracts oxidative stress, and is accomplished by the chemical affinity of the dopamine ligands within the C60-GSH-DOPA clusters 72, 73. The provision of C60 fullerene as a multifunctional center for these chemistries, in addition to the role of C60 as a hydrogen storage functional group, helps to create local reducing conditions suitable for prophylactic neural protection from oxidative stress induced degradation.

In the cellular lysosomes and other cellular vesicles, hydrogen protons 76 are exchanged with sodium 74, potassium 75, and other cations by various endogenous early endosome sodium-hydrogen exchangers such as NHE6. Medical evidence of genetic defects in the proteins used to traffic these cations are associated with the oligomeric agglomeration of plaques such as found in Alzheimer's Disease, some forms of autism, and Christianson syndrome; alpha synuclein is yet another oligomer that has confirmed salt bridges in Parkinson's disease. Alpha-synuclein needs to be present as individual fibrils to transport cations to biological membranes. A multiplicity of salt bridge hydrogen bonds are represented by the dotted lines 77 to bind the oligomer fibrils together so that they may no longer perform their cation shuttling function. C60-GSH-DOPA functions to artificially accelerate the trafficking of cations for proton exchange using a prosthetic pathway that prevents salt accumulation among the oligomeric fibrils, disassembles the oligomeric plaques formed by salt cations, and extracts the salt cations 74, 75 from alpha synuclein so that cations may not serve as salt bridges. The clusters of C60-GSH-DOPA 72, 73 constitute a prosthetic dual neurotransmitter having properties of both GSH and DOPA to enable the neural disease treatment of this composition according to the teachings of the present invention.

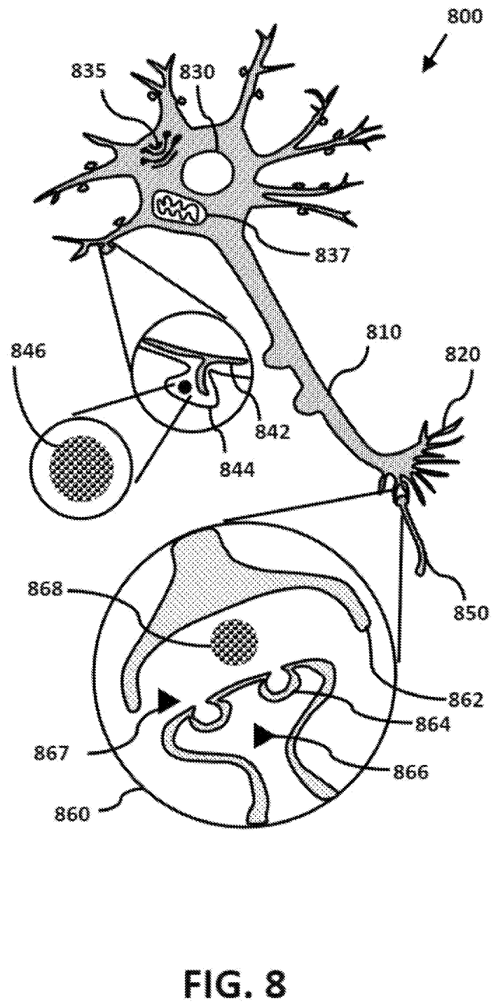

FIG. 8 illustrates the role of alpha-synuclein at a synapse and at some of the organelles of a neuron 800. It is well understood that alpha-synuclein binds to and regulates the transfer of calcium ions, especially those that are pooled and clustered within the synaptic vesicles 864 during neurotransmitter release 867 at the synaptic junction 860 between two neurons 810, 850. Alpha synuclein also influences the regulation of the vesicle trafficking from the endoplasmic reticulum 842 to the cell membrane at dendrites 844, and in vesicle adhesion to the Golgi complex 835 and neural cell nucleus 830. Alpha-synuclein localizes at the mitochondrial membranes 837, where it mitigates the effects of oxidative stress. These functions are enabled by the free radical, antioxidant, hydrogen proton storage, and cation trafficking composition of the C60-GSH-DOPA clusters 846, 868 that complement endogenous cation porter molecules in the manner of neurotransmitters, and thereby act to maintain the non-plaque independent fibril form of alpha-synuclein, as well as to establish cellular homeostasis among neurons.

Filopodia 820 are slender cytoplasmic neural projections that extend beyond a first neuron 810 and may have at least one synaptic junction 860 with a second neuron illustrated as a partial section of another filopodium extension 850. At least one metabolized C60-GSH-DOPA cluster 868 has been reduced in size to about less than 35 nanometers as part of the metabolic process, which enables it to enter the synaptic cleft between 864 and 862. Cluster 868 provides multifunctional roles to stabilize the membrane lipid interaction at the synaptic junction 860 where neurotransmitter 866 accumulates within the presynaptic terminal as neural bouton 864 for release into the synaptic gap 867 to be received by neural receptors at the proximal neuron providing the post synaptic terminal 862. Vesicles such as 864 may detach and travel with neurotransmitter 867 while carrying charged cations such as Na+ and Ca+2, wherein independent alpha-synuclein fibrils are critical to maintain the multiplicity of cations as adducts. The redox chemistry homeostasis provided by C60-GSH-DOPA clusters 846, 868 destabilizes plaques by the prevention of the free-radical and oxidative kinetics of alpha-synuclein aggregation, and by extracting cations from between alpha-synuclein fibrils, thereby halting or reversing the formation of oligomeric protein aggregates and their associated toxicity, according to the teachings of the present invention.

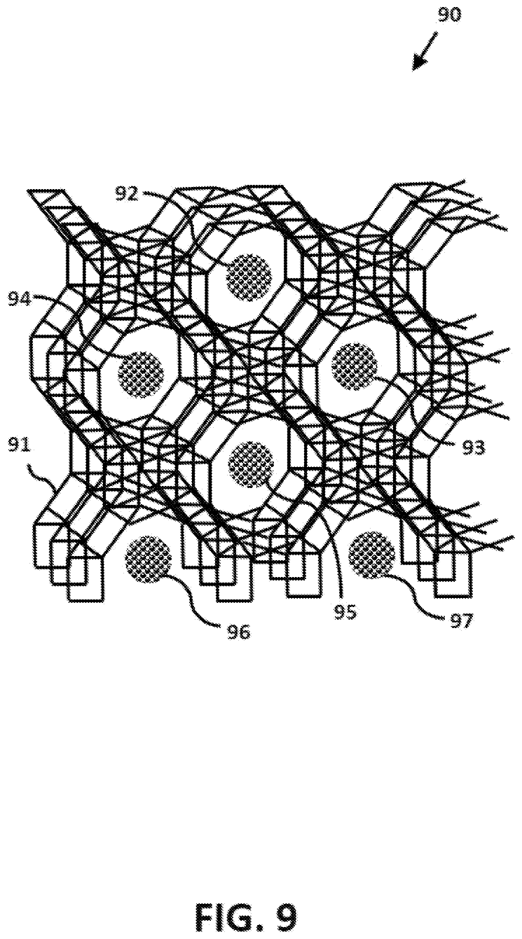

FIG. 9 illustrates a zeolite impregnated with a C60-GSH-L-dopa 90. Transcarpathian zeolite (clinoptilolite) 91 is a type of mineral provided with a highly negative charged network structure achieving a system of reproducible and well-defined pores and channels. Clinoptilolite zeolite 91 is well known to adsorb oppositely charged nitrogen containing compounds including protonated ammonia and protonated amino acids which serve as positive counter-ion and hydrogen bonding adducts with the composition of C60-GSH-L-dopa in the form of clusters 92, 93, 94, 95, 96, and 97 having sizes sufficiently small to fit within the mineral scaffold, where the channels therein can typically range from greater than 100 nanometers to less than about 5 microns in size. It is also known that at pH greater than 7, as well as under saline or physiological ionic salt conditions, clinoptilolite zeolite displaces and expresses the positively charged nitrogen compounds and counterions stored within the channels of zeolite 91. The salt and pH moderated regenerant property of clinoptilolite 91 towards reversible expression and delivery or release of positive charged nitrogen compounds has led to the widespread economic commercial adoption of clinoptilolite Transcarpathian zeolite 91 as a dietary supplement. It is therefore specified to utilize this ion-exchange property of zeolite 91 as one exemplary way to perform timed release of the C60-GSH-L-dopa composition of the present invention as a practical and cost-effective method of delivering this composition by oral administration, according to the teachings of the present invention.

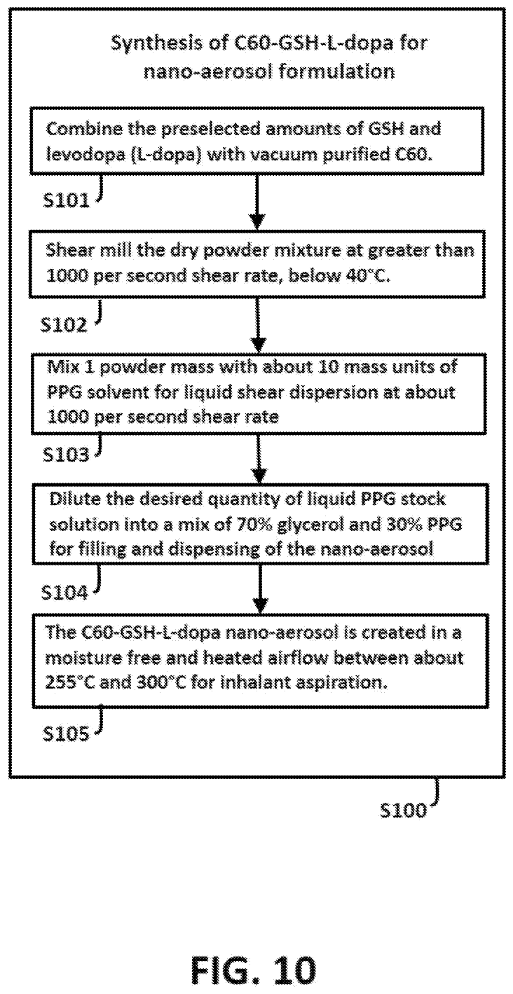

FIG. 10 is a flowchart representation of a synthesis and nano-aerosol formulation of C60-GSH-L-dopa 100. In step 101 at least about one molar equivalents of pure glutathione (GSH) is combined with one molar equivalent of vacuum purified buckminsterfullerene (C60) and at least about one and nominally 2 molar equivalents of pure levodopa (L-dopa). In step 102, the dry powder mixture is reactive shear milled at greater than 1000 per second shear rate at a processing temperature maintained below 40° C. to minimize the covalent bonding of amine groups from the GSH onto the C60, while maximizing the pi-carbonyl and pi-aromatic bonding with C60. One way that low temperature processing can be accelerated at higher shear rates for less time, is to provide an oxygen free processing atmosphere. In step 103 the sheared C60-GSH-L-dopa product is added to polypropylene glycol (PPG) solvent in a 1:10 mass ratio of dry powder to solvent for liquid shear at about 1000 per second shear rate to full product dissolution. In step 104, the desired concentration of C60-GSH-L-dopa is created by dissolving a volumetric amount of the C60-GSH-L-dopa solution into a solvent mixture of glycerol with polypropylene glycol to achieve the desired final concentration of between about 20 ppm and 2000 ppm to obtain a suitable vaporized inhalant or a dosage for nano-aerosol inhalant delivery. This final dilution solvent mixture comprises about 70% glycerol and 30% polypropylene glycol by volume. All solvated components for dispensing are to be kept free of moisture in a quality-controlled process. In step 105, a metered amount of the nano aerosol is generated by a commercially available electronic dispensing device, such as by heating the formulated fluid at from about 255° C. up to about 300° C., but no greater than about 300° C. to avoid oxidation or breakdown of the nano-aerosol, and to maintain temperatures suitable for client aspiration, according to the teachings of the present invention.

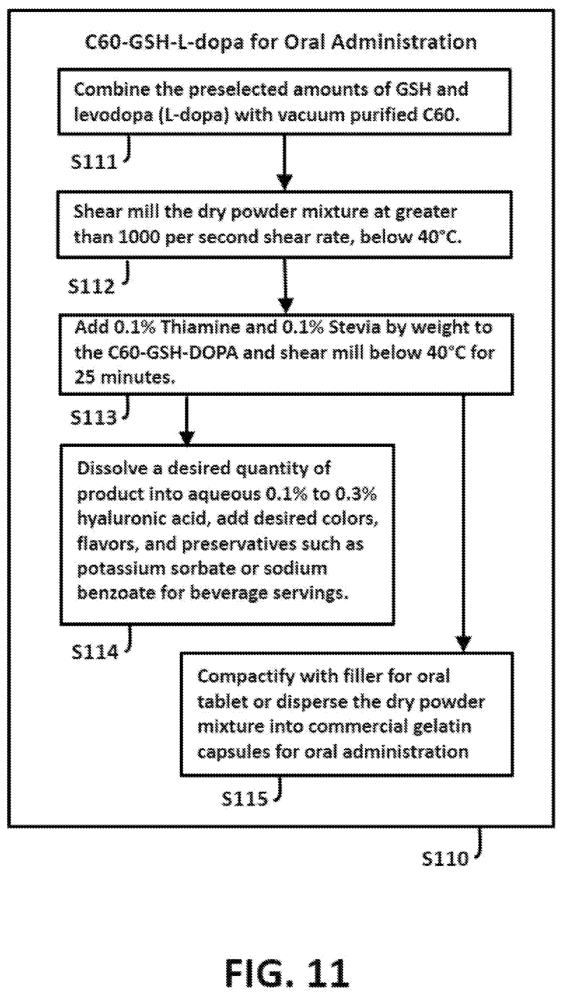

FIG. 11 is a flowchart representation of a synthesis of C60-GSH-L-dopa and a formulation for Oral Administration 110. In step 111 at least about one molar equivalents of pure glutathione (GSH) is combined with one molar equivalent of vacuum purified buckminsterfullerene (C60) and at least about one and nominally 2 molar equivalents of pure levodopa (L-dopa). In step 112 the dry powder mixture is shear milled at greater than 1000 per second shear rate, the processing maintained at a temperature below 40° C. to minimize the covalent bonding of amine groups from the GSH onto the C60, while maximizing the pi-carbonyl and pi-aromatic bonding with C60. One way that low temperature processing can be accelerated at higher shear rates for less time, is to provide an oxygen free processing atmosphere. In a first alternative step 114, a desired quantity of hydrogen bonded C60-GSH-L-dopa powder product obtained from step 113 is dissolved into aqueous 0.1% to 0.3% hyaluronic acid, then desired colors, flavors, and preservatives such as potassium sorbate or sodium benzoate are added for oral administration or beverage servings. In a second alternative step 115, the C60-GSH-L-dopa powder product is combined with one or more pharmaceutically acceptable carriers like suitable USP food grade binders as delivery materials in any combination. These carriers and delivery materials are generally known as excipients and fillers, of which non-limiting examples include commercially available calcium carbonate, zeolite, methyl cellulose, and gel peptides for placement into a compressed tablet or a gel capsule as desired for oral administration, according to the teachings of the present invention.

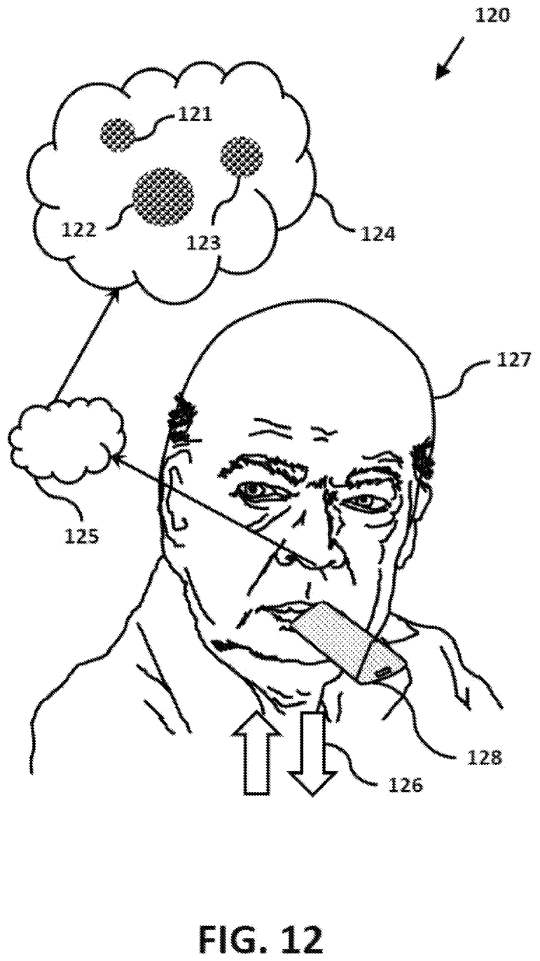

FIG. 12 illustrates a personal administration method 120 for an aspirated nano-aerosol delivery system containing an C60-GSH-L-dopa composition. The nano-aerosol generating device filled with C60-GSH-L-dopa dispensing solution 128 is provided for dispersing the inhalant gas wherein the nano-particles are and nebulized. The dispensing method of commercially available device 128 may also be more commonly known as a nebulizer, or an electronic vaporizing device, or an electronic cigarette, or the functional part of a hookah to be shared among several users. In all cases these systems serve to carry the C60-GSH-L-dopa in a carrier fluid dispenser 128, move that composition in nebulized form along with an aerosolized solvent, and transfer this composition in substantially gaseous dispersion to the nose, mouth, trachea, and airways of a patient or user 127. One intended use of the C60-GSH-L-dopa composition is to treat, delay or arrest the incidence of Parkinson's disease (PD), Alzheimer's disease (AD), and other cognitive disorders wherein the nano-aerosol can expedite targeted delivery to the brain by avoiding a passage through the digestive system.

Some of the nano-aerosolized composition is exhaled and shown as particulate clusters 121, 122, 123 within exhaled smoke puffs 124 and 125 emitted on exhalation as indicated by the direction of thin line arrows radiating away from the nose of the subject 127. Delivery of the C60-GSH-L-dopa nano-aerosol composition from dispenser 128 provides antioxidant properties to the mucus airway tissues wherein destruction of free radicals and oxidants associated with motor neuron disease and Parkinson's disease are part of the treatment and alpha-synuclein plaque mitigation is provided using this method. Systems that may be used for the method of dispersion of the C60-GSH-L-dopa represented by exemplary device 128, include, without limitation, any of the electronic cigarette devices produced internationally and listed in Appendix 4.1, “Major E-cigarette Manufacturers” of the “2016 Surgeon General's Report: E-Cigarette Use Among Youth and Young Adults” published by the Center for Disease Control and Prevention (CDC), Office of Smoking and Health (OSH) freely available at the CDC.GOV website, and/or any combination of piezoelectric, resistively heated, or inductively heated vaporized fluid delivery methods that can be utilized to deliver the composition of the present invention, especially when approved as a medical drug delivery device. Each embodied variation of such methods without limit are intended to aspirate aerosols as the method of therapeutic substance delivery of the composition of the present invention directed into the nasal cavities, mouth, tracheal breathing orifice, or intubated trachea of a patient. The supply direction of nebulized feed of C60-GSH-L-dopa on inhalation and exhalation are delivered into the airways and lungs of the intended patient by the flow of supplied air as indicated by the direction of upward and downward facing large white arrows 126, when used according to the teachings of the present invention.

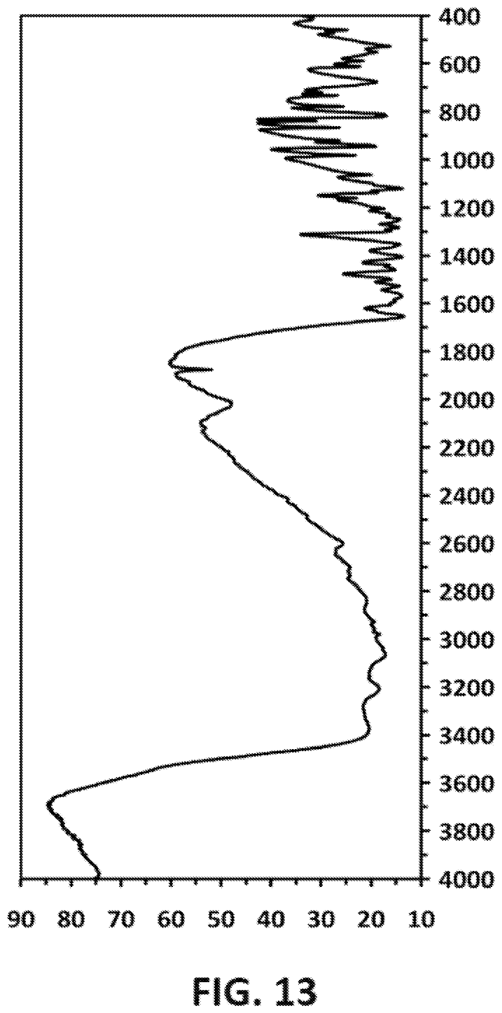

FIG. 13 illustrates experimental FTIR data for levodopa. All the Fourier transform infra-red (FTIR) spectrographs hereinafter were measured by transmittance using the potassium bromide (KBr) compressed flow solid pellet compact preparation method. The material used for analysis was obtained by the method of mixing, crushing, and consolidating under 7 metric tons of pressure, about 0.001 grams of the analyte substance with 1 gram of a diluent solid KBr that is substantially transparent to infrared light, and which flows under pressure to form a translucent pellet of about 0.4 mm thickness. Spectral background subtraction in air using a control pellet of the same mass and thickness having pure KBr was used to obtain a baseline instrument infrared spectral response. This method is generally referred to as the ‘KBr pellet’ sample preparation method, and it is used hereinafter throughout for each FTIR experimental data collection and spectral analysis. The Fourier transform infrared spectrophotometer used herein to obtain FTIR spectra throughout, is a model RF6000 FTIR instrument manufactured by Shimadzu of Japan. Each FTIR data graph hereinafter is provided with a numeric scale ranging from 400 to 4000 to represent reciprocal centimeters or (cm−1) in wavenumbers.

The numeric scale ranging from 10 to 90 represents percentage transmittance and has units of %. The FTIR absorbance peak at 3359 cm−1 is attributed to the amine nitrogen-hydrogen vibration (N—H). At 3200 cm−1 appears an oxygen-hydrogen (O—H) stretching vibration, and at 3046 cm−1 is an aromatic hydrogen stretching vibration. The primary amine functional group is indicated by the two (N—H) bending absorbance vibration bands at 1653 cm−1 and at1567 cm−1. The peaks between 1064 cm−1 and 1200 cm−1 are due to (C—N) stretching vibrations. The sharp and intense peak at 817 cm−1 indicates the N—H bending vibration. There is evidence of a band at about 1500 cm−1 that can be attributed to the C═C bond in the benzene ring structure. Comparison of the illustrated experimental FTIR data for levodopa 1200 indicates similarity to the FTIR absorbances reported for levodopa that are available from the scientific literature, and may be used for confirmation of the raw material composition according to the teachings of the present invention;

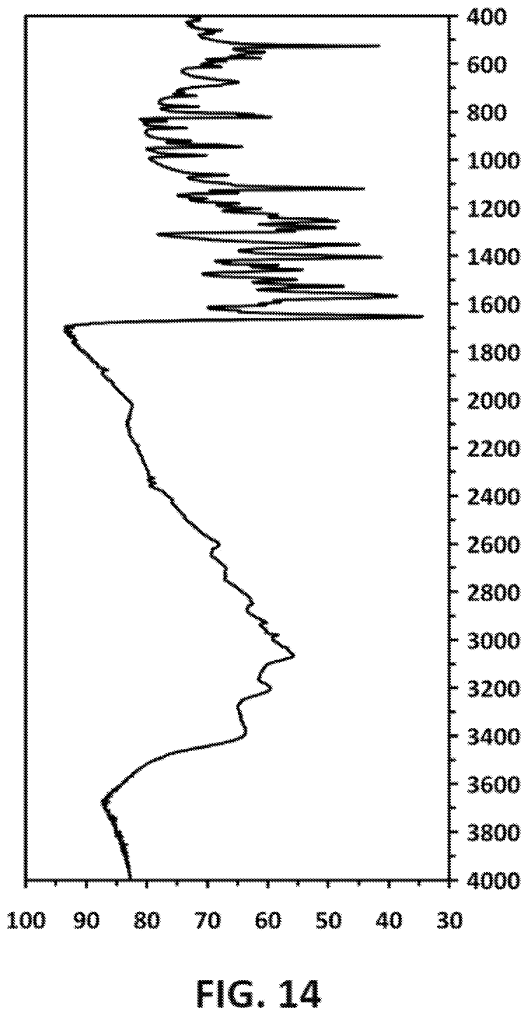

FIG. 14 illustrates experimental FTIR data for fullerene C60 reacted with levodopa, being C60-Ldopa. The numeric scale ranging from 30 to 100 represents percentage transmittance and has units of %. The characteristic strong and sharp buckminsterfullerene (C60) aromatic carbon-carbon stretching band is present at 526 cm−1. The FTIR absorbance peak at 3373 cm−1 is attributed to the amine nitrogen-hydrogen vibration (N—H). At 3192 cm−1 appears an oxygen-hydrogen (O—H) stretching vibration, and at 3062 cm−1 is an aromatic hydrogen stretching vibration. The two bands arising from the primary amine functional group are indicated by the (N—H) bending absorbance vibrations and remain unchanged at 1653 cm−1 and at1567 cm−1, confirming that there was no chemical reaction to alter the amine functional group. The peaks between 1064 cm−1 and 1200 cm−1 are due to (C—N) stretching vibrations. The sharp and intense peak at 821 cm−1 indicates the N—H bending vibration. The observed reduction of absorbance intensity in the region of 1450 cm−1 to 1500 cm−1 can be attributed to the attenuation of the C═C bond vibrations in the levodopa benzene ring that has become sterically constrained by aromatic-pi bonding with the C60 functional group, wherein the spatially confined geometry impacts the bond mobility in bending and stretching modes associated with the formation of the aromatic pi to aromatic pi bonds.

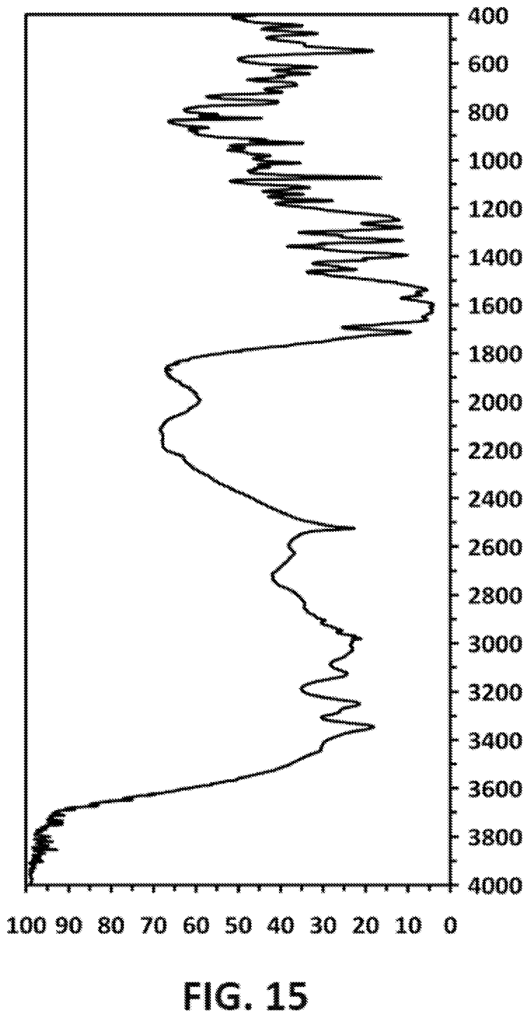

FIG. 15 illustrates experimental FTIR data for GSH raw material that was used to synthesize the compositions of the present invention. The numeric scale ranging from 0 to 100 represents percentage transmittance and has units of %. The characteristic reduced glutathione sulfhydryl (—S—H) peak is observed at 2523 cm−1. The peaks at 2980 cm−1 and 1455 cm−1 arise from the stretching and bending vibrations of aliphatic C—H group in glutathione. The peak at 1276 cm−1 is a tertiary amide peak, and the very sharp absorbance peak at 1073 cm−1 provides a characteristic carbon nitrogen (C—N) stretch. The strong and sharp peak observed at 1713 cm−1 and the one at 1393 cm−1 are attributed to a deprotonated carboxylic acid (—COO), where the former is a symmetric vibration, and the latter is an asymmetric vibration mode of this functional group. The overall infrared absorbance spectral features are consistent with and indicate chemical similarity to reduced glutathione as may be found in published public FTIR spectra, according to the teachings of the present invention.

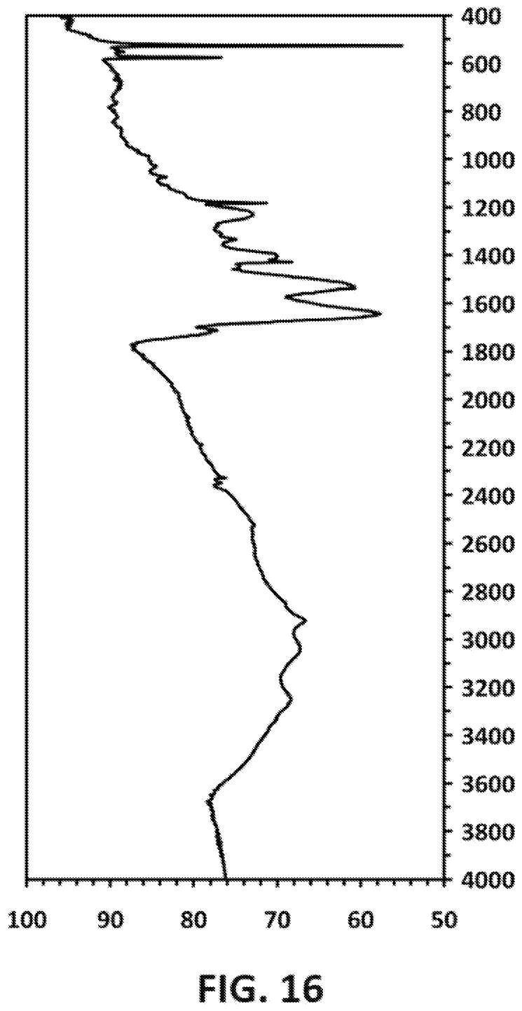

FIG. 16 illustrates experimental FTIR data for the intermediate compound fullerene glutathione (C60-GSH). The numeric scale ranging from 50 to 100 represents percentage transmittance and has units of %. In comparison with FIG. 15, it is notable that the reduced glutathione sulfhydryl (S—H) peak is no longer observed at 2523 cm−1, indicating here that the sulfur-hydrogen stretch has disappeared because of the chemical reaction of GSH with C60. This observation supports the molecular sulfur binding reaction of FIG. 3. Notable also is the very strong and sharp C60 fullerene aromatic carbon-carbon stretching bands at 576 cm−1 and 526 cm−1.

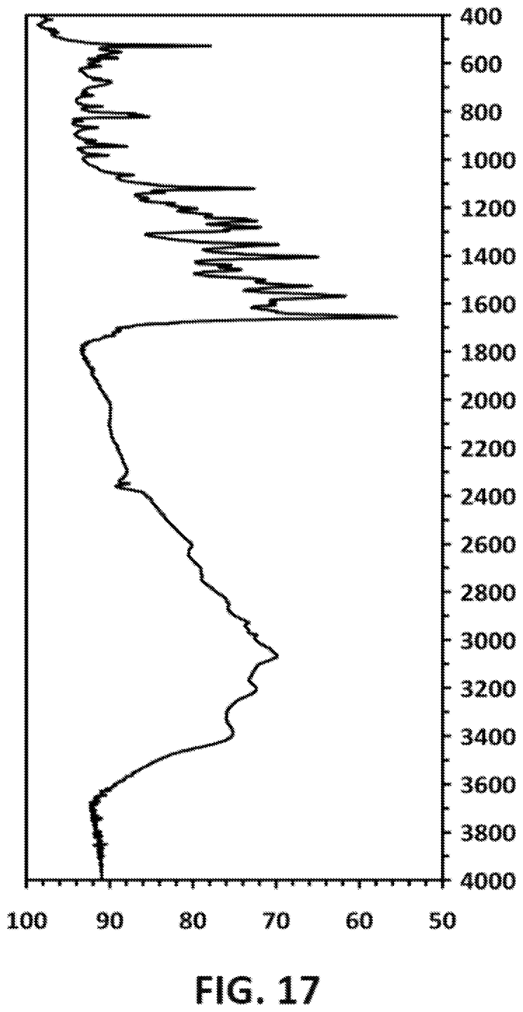

FIG. 17 illustrates experimental FTIR data for the final product C60-GSH-L-dopa. The numeric scale ranging from 0 to 100 represents percentage transmittance and has units of %. The absorption peak at 526 cm−1 is characteristic for C60 fullerene carbon-carbon (C═C) bonds. The sulfhydryl (S—H) vibration absorbance previously observed at 2523 cm−1 for the free glutathione in FIG. 16 was not observed for C60-GSH-DOPA experimental results. This indicates that the glutathione was probably deprotonated and coordinated to the surface of the fullerene (C60) nanoparticle through the sulfur and suggests that glutathione caps the fullerene nanoparticles through the thiol from the cysteine portion of the glutathione ligand. The characteristic primary amine group of levodopa or dopamine obtains a clearly resolved absorbance peak at 3389 cm−1; this indicates that these molecular groups are pi-bonded to the aryl regions of fullerene, leaving their primary amine function available for the intended interaction with cellular components. This confirms the absorbances of levodopa (L-dopa) and GSH interacting as functional groups in combination with C60 by FTIR as a distinguishable material having clearly recognizable chemical signatures that are characteristic of the composition of the present invention.

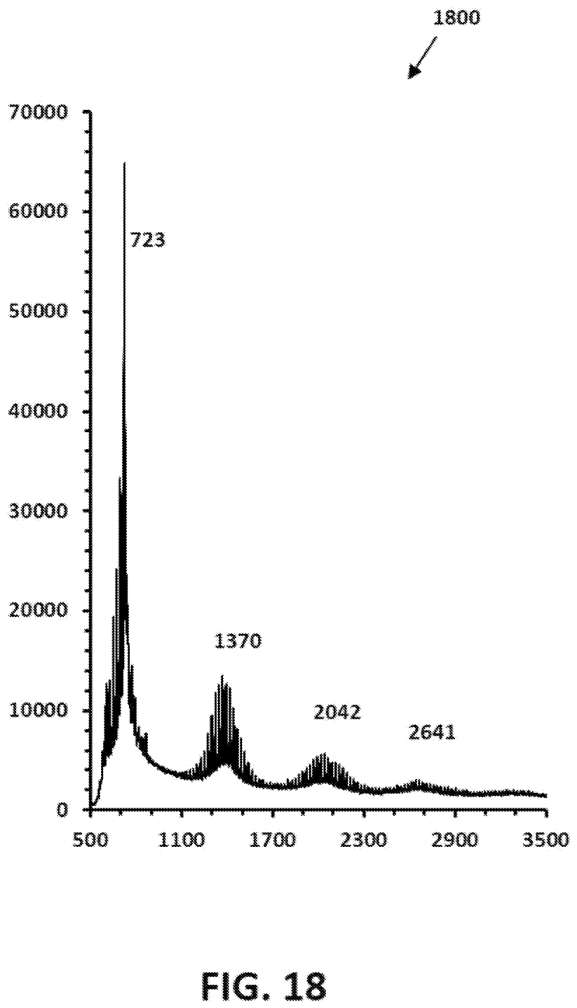

FIG. 18 illustrates experimental negative mode MALDI-TOF mass spectrograph data for C60-L-dopa material 1800. This sample, as well as each of the subsequent MALDI-TOF experimental test results hereinafter, was introduced for test by laser vaporization into a Voyager Mass Spectrograph from Applied Biosystems (Foster City, Calif., USA). Negative mode bombardment was by fast moving electrons at about 70 eV energy. This resulted in molecular fragmentation and electron removal from the highest molecular orbital energy as molecular ions were formed. The ratio of mass to charge (m/z) is used to determine the molecular ion fragments to help determine the pieces of the original molecule in this assay. The mass peak at 723 m/z corresponds to the molecular ion fragment of fullerene C60 of mass 720 having three adducted hydrogen atoms. The very broad mass peaks at 1370, 2042, and 2641 are attributed to indicate predominantly dimeric and some trimeric C60 chains appended to each other and to interstitial levodopa by pi-pi bonding. The rider peaks on the broader peaks indicate the loss of small ion fragments such as those having a mass of 17 from (—OH) hydroxyl groups. The overall experimental test results characterize the molecular ion breakdown products of C60-L-dopa, where C60-L-dopa may be used to further synthesize the composition of the present invention.

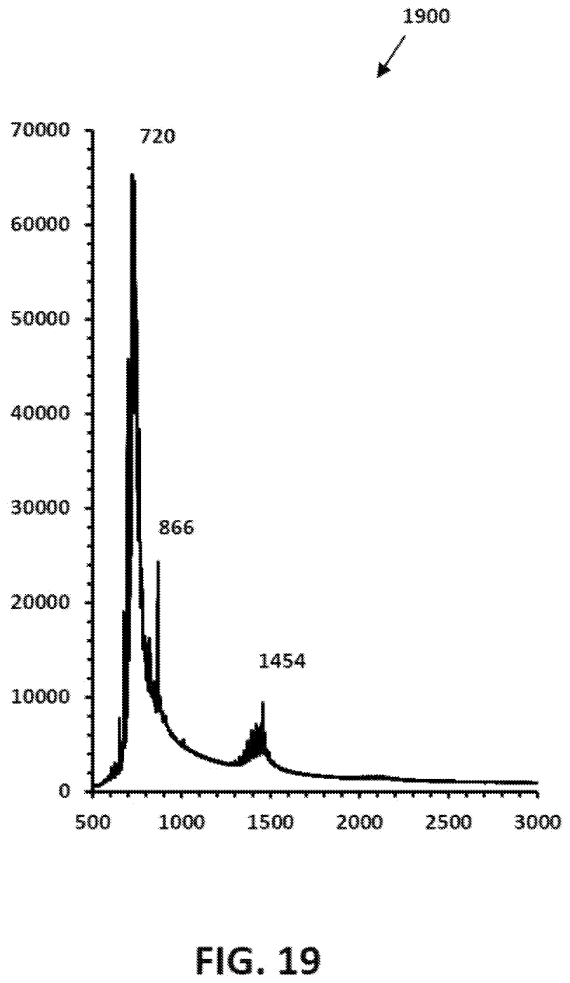

FIG. 19 illustrates experimental negative mode MALDI-TOF mass spectrograph data for C60-GSH material 1900. This test sample resulted from reacting an equivalent molar quantity of glutathione to the molar equivalent of pristine fullerene C60. The largest peak observed was the primary and core molecular ion, this being a fullerene ion as indicated by the numeric peak label at mass to charge ratio of 720. The primary molecular ion was subsequently verified using a pristine pure reference material of C60 tested immediately after this test, under both negative mode and positive mode test conditions (results are not shown here). The observed molecular fragment at 866 is characteristic for a fullerene C60 obtaining a residual spallation fragment from glutathione that was incompletely removed. The cluster of peaks with a maximum at 1454 is attributed to C60-GSH, wherein one molecular mass of glutathione is bonded to one molecular mass of buckminsterfullerene, and characterizes the C60-GSH component that may be used to further synthesize the composition of the present invention.

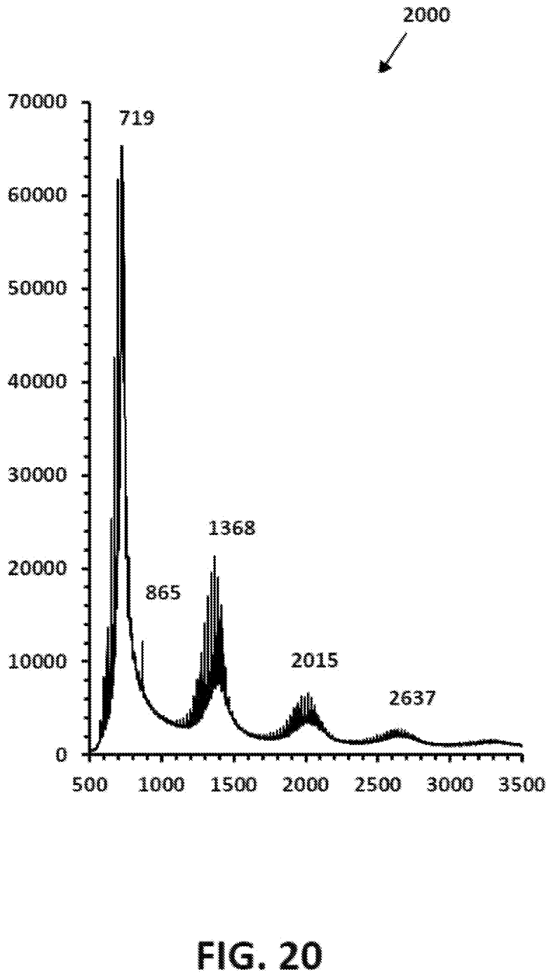

FIG. 20 illustrates experimental negative mode MALDI-TOF mass spectrograph data for C60-GSH-DOPA 2000. The largest peak observed is the molecular ion fragment for C60 fullerene as indicated by the mass to charge ratio of 719. The characteristic glutathione ion spallation fragment of 866 in FIG. 19 is also seen illustrated here at 865 mass to charge ratio. The first broad cluster of peaks present at 1368 m/z is like the result of 1370 m/z found for C60-DOPA in FIG. 20. The broad cluster of peaks at 2015 and 2637 m/z are attributed to dimeric and trimeric molecular ion fragments of C60-GSH-DOPA, where a loss of mass attributed to the decarboxylation or removal of some of the carboxylic acid functional group from the dimer or trimer can explain the mass reduction in these fragments. The overall illustrated mass spectral fingerprint of molecular ion fragments 2000 characterizes C60-GSH-DOPA according to the teachings and composition of the present invention.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments but defined in accordance with the foregoing claims appended hereto and their equivalents.

What is claimed is:

1. A compound comprising:
a buckminsterfullerene C60 bonded to a first neurotransmitter.

2. The compound of claim 1 further comprising a second neurotransmitter bonded to the buckminsterfullerene C60 and different than the first neurotransmitter.

3. The compound of claim 1 wherein the buckminsterfullerene C60 is bonded to the first neurotransmitter by a pi bond.

4. The compound of claim 2 wherein the buckminsterfullerene C60 is bonded to the first neurotransmitter by a first pi bond and the buckminsterfullerene C60 is bonded to the second neurotransmitter by a second pi bond.

5. The compound of claim 1 wherein the first neurotransmitter comprises glutathione.

6. The compound of claim 2 wherein the first neurotransmitter comprises glutathione and the second neurotransmitter comprises either levodopa or dopamine.

7. The compound of claim 1 disposed within a zeolite.

8. A method of curing, treating, or prophylactically avoiding motor neuron dysfunction related to oligomeric alpha-synuclein plaque formation in Parkinson's disease and Lewy Body Disease in a subject, or prophylactically avoiding motor neuron dysfunction related to oligomeric plaque formation in Alzheimer's disease or Amyotrophic Lateral Sclerosis (ALS) in the subject, comprising the step of:
administering to the subject an effective amount of a compound including a buckminsterfullerene C60 bonded to a first neurotransmitter and a second neurotransmitter different than the first neurotransmitter.

9. The method of claim 8 wherein the first neurotransmitter comprises glutathione and the second neurotransmitter comprises either levodopa or dopamine.

10. The method of claim 8 wherein administering the compound comprises administering a composition containing the compound in a pharmaceutically acceptable carrier.

11. The method of claim 10 wherein the composition comprises a tablet, capsule, pill, powder, granule, or a form suitable for injection.

12. The method of claim 10 wherein the pharmaceutically acceptable carrier comprises a zeolite.

13. The method of claim 8 wherein administering the compound comprises administration by an intravenous, intramuscular, subcutaneous, intrathecal, intraperitoneal, topical, nasal, or oral route.

14. The method of claim 8 wherein an oral dosage comprises up to about 500 mg of the compound.

15. The method of claim 8 wherein administering the compound comprises intramuscular, intravenous, or subcutaneous administration in an amount of from about 0.1 mg/Kg to about 5 mg/Kg.

16. The method of claim 8 wherein administering the compound comprises administration by a nano aerosol, a vapor, a powder, a dust, or an aerosolized inhalant.

17. A method of making a C60 bonded to a neurotransmitter, the method comprising:
bonding a glutathione to the C60; and
bonding either a levodopa or a dopamine to the C60.

18. The method of claim 17 wherein bonding the glutathione to the C60 and bonding either the levodopa or the dopamine to the C60 are performed at no more than 40° C.

19. The method of claim 17 wherein bonding the glutathione to the C60 and bonding either the levodopa or the dopamine to the C60 is performed by reaction shear mixing.

20. The method of claim 17 wherein bonding the glutathione to the C60 and bonding either the levodopa or the dopamine to the C60 is performed in an oxygen-free environment.

21. The method of claim 17 wherein bonding the glutathione to the C60 and bonding either the levodopa or the dopamine to the C60 are performed together.

22. The method of claim 17 further comprising disposing the buckminsterfullerene C60 bonded to the glutathione and either the levodopa or the dopamine within channels of a zeolite.

23. The method of claim 17 further comprising combining the buckminsterfullerene C60 bonded to the glutathione and either the levodopa or the dopamine with a pharmaceutically acceptable carrier.

24. The method of claim 17 further comprising adding the buckminsterfullerene C60 bonded to the glutathione and either the levodopa or the dopamine to a mixture of glycerol and polypropylene glycol.

25. The method of claim 17 further comprising dissolving the buckminsterfullerene C60 bonded to the glutathione and either the levodopa or the dopamine, into a hyaluronic acid solution.
